(19) United States
(12) Patent Application Publication
(10) Pub. No.: US 2011/0318205 A1
(43) Pub. Date: Dec. 29, 2011

(54) SOLVENT DELIVERY PUMP

(75) Inventor: Yoshiaki Aso, Kyoto (JP)

(73) Assignee: SHIMADZU CORPORATION, Kyoto (JP)

(21) Appl. No.: 13/164,283

(22) Filed: Jun. 20, 2011

(30) Foreign Application Priority Data
Jun. 29, 2010 (JP) 2010-147029

(51) Int. Cl. A61M 1/00

(52) U.S. Cl. 417/437

(57) ABSTRACT

A solvent delivery pump is provided, which includes: a cylindrical pump chamber, an inside of which is made of metal; a plunger inserted from a tip end into the pump chamber to reciprocate in a certain direction to increase and decrease a capacity in the pump chamber; a capacity decreasing member made of resin and covering at least a portion of an inner wall of the pump chamber to reduce a clearance from the plunger; and a seal member provided to a plunger insertion portion of the pump chamber to come in sliding contact with an outer peripheral face of the plunger to seal the pump chamber. Preferable examples of the capacity decreasing member are a cylindrical sleeve covering at least a portion of an inner peripheral face of the pump chamber, a spacer disposed on an innermost face of the pump chamber to reduce a clearance from a tip end face of the plunger which has reached a top dead center, or both of the sleeve and the spacer.

### BACKGROUND OF THE INVENTION

1. Field of the Invention

The present invention relates to a solvent delivery pump for sucking and discharging a solution by reciprocating a plunger in a pump chamber.

2. Description of the Related Art

A common plunger-type solvent delivery pump sucks a solution into a pump chamber and discharges the sucked solution with a plunger reciprocating in the cylindrical pump chamber to vary a capacity in a space in the pump chamber. In general, sapphire is used as material of the plunger, and stainless steel is used as material of the pump chamber.

In such a solvent delivery pump, a space between an inner wall of the pump chamber and an outside shape of the plunger is designed to be as small as possible when the plunger has reached a top dead center (when discharge of the solution has been finished and sucking of the solution is to be started). If there is a large space between the inner wall of the pump chamber and the outside shape of the plunger, an amount of solution receiving compression (compression capacity) when the plunger is at the top dead center is large, which causes pulsation of solution sending. If the compression capacity in the pump chamber is large, time taken for an interior of the pump chamber to return to atmospheric pressure at the time of sucking of the solution becomes long and high pressure is applied on a seal member for a long time to reduce durability of the seal member.

In particular, a seal member in such a shape as to improve sealing performance of a plunger insertion portion of a pump chamber by utilizing pressure in the pump chamber is recently used in some cases (see Japanese Patent Application Laid-Open No. 2008-180088). If the high pressure is applied on such a seal member for an extended amount of time, it significantly shortens life of the seal member.

However, if a clearance between the inner wall of the pump chamber and the outside shape of the plunger is excessively small, the outside shape of the plunger and the inner wall of the pump chamber may come in contact with each other. If the plunger made of sapphire or the like and the inner wall of the pump chamber made of metal such as stainless steel come in contact with each other, generation of frictional heat shortens the life of the seal member and contact of a tip end of the plunger with an innermost portion of the pump chamber causes damage to the tip end of the plunger. Therefore, it is necessary to provide a clearance which can at least avoid contact between the inner wall of the pump chamber and the outside shape of the plunger. As described above, there is a limitation to reduction of size of the space between the inner wall of the pump chamber and the outside shape of the plunger.

### SUMMARY OF THE INVENTION

it is therefore an object of the present invention to decrease a compression capacity of a solution in a pump chamber without bringing a metal inner wall of a pump chamber and an outside shape of a plunger in contact with each other.

A solvent delivery pump according to the invention includes: a pump chamber; a plunger inserted from a tip end into the pump chamber to reciprocate in a certain direction to increase and decrease a capacity in the pump chamber; and a seal member provided to a plunger insertion portion of the pump chamber to come in sliding contact with an outer peripheral face of the plunger to seal the pump chamber. An inside of the pump chamber is cylindrical and made of metal and a capacity decreasing member made of resin and covering at least a portion of an inner wall of the pump chamber to reduce a clearance from the plunger is provided.

As material of the capacity decreasing member, there are wholly aromatic polyimide resin and polyether ether ketone (hereafter, PEEK) resin. These resins are excellent in wear resistance.

As an example of the capacity decreasing member, there is a cylindrical sleeve covering at least a portion of an inner peripheral face of the pump chamber. In this case, an inner diameter of the sleeve and a dimension of an outer peripheral face of the plunger are designed so that the clearance between the inner diameter of the sleeve and the outer peripheral face of the plunger becomes as small as possible, e.g., a clearance of about 10 to 100 μm. With such a minute clearance, the plunger is expected to come in contact with the sleeve in reciprocation of the plunger. However, since the resin material is superior in sliding property to metal material, little frictional heat is generated when the sleeve comes in contact with the outer peripheral face of the plunger, and the seal member is less likely to be deteriorated by the frictional heat even if the solution is sent with the inner wall of the pump chamber and the outer peripheral face of the plunger in contact with each other.

Another example of the capacity decreasing member is a spacer covering an innermost face of the pump chamber to fill a clearance from a tip end face of the plunger which has reached a top dead center.

In each case, it is possible to decrease a compression capacity in the pump chamber to suppress a load applied on the seal member. If both the sleeve and the spacer are provided, it is possible to further decrease the compression capacity.

In operation of the plunger, a state in which the solution is sucked into the pump chamber to a maximum degree (a state in which the plunger is fully pulled from the pump chamber) is referred to as a “bottom dead center” and a state in which the solution in the pump chamber is discharged to a maximum degree (a state in which the plunger is fully inserted into the pump chamber) is referred to as the “top dead center”.

As described above, because the solvent delivery pump in the invention is provided with the resin capacity decreasing member covering at least the portion of the inner wall of the pump chamber to fill the clearance from the plunger, it is possible to decrease the compression capacity in the pump chamber to shorten time for which pressure in the pump chamber is high to reduce the load applied on the seal member to thereby prevent shortening of life of the seal member.

### BRIEF DESCRIPTION OF THE DRAWINGS

FIG. 1A is a sectional view of a first example of a solvent delivery pump and FIG. 1B is an enlarged sectional view of a pump chamber and a portion around it in the example.

FIG. 2A is a sectional view of a second example of the solvent delivery pump and FIG. 2B is an enlarged sectional view of a pump chamber and a portion around it in the example.

FIG. 3A is a sectional view of a third example of the solvent delivery pump and FIG. 3B is an enlarged sectional view of a pump chamber and a portion around it in the example.

FIG. 4 is a flow path diagram showing en example of a liquid chromatograph including the solvent delivery pump in the first, second or third example.

### DETAILED DESCRIPTION OF THE INVENTION

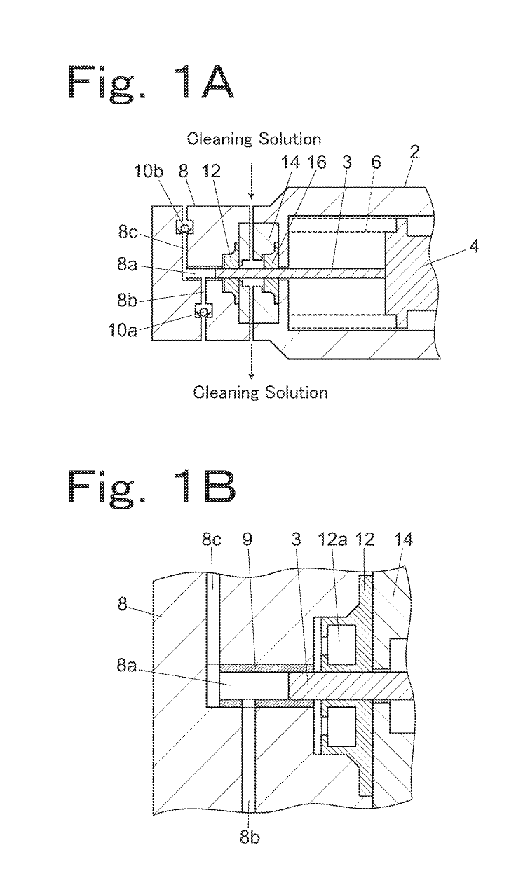

A first example of a solvent delivery pump will be described by using FIGS. 1A and 1B. The solvent delivery pump in the example includes a syringe 2 and a pump head 8. The syringe 2 houses in itself a cross head 4. The cross head 4 retains an end face on a base end side of the plunger 3 and is pushed against a peripheral face of a cam (not shown) by an elastic force of a spring 6. When the cam is rotated by a driving motor (not shown), the cross head 4 and the plunger 3 reciprocate following the peripheral face of the cam.

The pump head 8 is mounted to the syringe 2. The pump head 8 includes a pump chamber 8a, a solution sucking flow path 8b, and a solution discharge flow path 8c so as to suck and discharge a solution by reciprocation of a tip end portion of the plunger 3 retained on the cross head 4. The solution sucking flow path 8b and the solution discharge flow path 8c are respectively provided with check valves 10a and 10b for utilizing changes in pressure in the pump chamber 8a to open and close these flow paths 8b and 8c to prevent back-flow.

The tip end portion of the plunger 3 is inserted into the pump chamber 8a and reciprocates in such a direction (a rightward direction in the drawing) as to suck the solution into the pump chamber 8a from a solution sucking flow path 8b while expanding a space in the pump chamber 8a and in such a direction (a leftward direction in the drawing) as to push the solution in the pump chamber 8a out into the solution discharge flow path 8c while narrowing the space in the pump chamber 8a as the cross head 4 reciprocates.

The pump chamber 8a is in a cylindrical shape and a sleeve 9 as a capacity decreasing member is secured to an inner peripheral face of the pump chamber 8a. Material of the sleeve 9 is, for example, Vespel (registered trademark) which is wholly aromatic polyimide resin or PEEK resin. An inner diameter of the pump chamber 8a is, for example, 2.2 mm, and an outer diameter of the plunger 3 is, for example, 2 mm. In this case, a wall thickness of the sleeve 9 is slightly smaller than 0.1 mm so that the sleeve 9 is fitted in the plunger 3 with a clearance of 10 to tens of micrometers between an inner side of the sleeve 9 and the plunger 3. A through hole is provided in a side wall portion of the sleeve 9 and the through hole and the solution sucking flow path 8b are aligned with each other so that the solution from the solution sucking flow path 8b can pass through the through hole.

As a method of providing the sleeve 9 in the pump chamber 8a, the sleeve 9 is press-fitted in the pump chamber 8a before forming the flow paths 8b and 8c, and then the flow paths 8b and 8c are formed. In machining for forming the flow path 8b, the hole is formed in the sleeve 9 as well in a position of the flow path 8b.

By providing the sleeve 9, a space created by a clearance between an inner wall of the pump chamber 8a and an outside shape of the plunger 3 becomes small in the pump chamber 8a. As a result, an amount of solution compressed when the plunger 3 has reached a top dead center (compression capacity) reduces, time taken for an inside of the pump chamber 8a to return to atmospheric pressure when the plunger 3 starts sucking operation is shortened, and time for which the inside of the pump chamber 8a is at high pressure is shortened. Therefore, a load applied on a plunger seal 12 reduces, and it is possible to prevent shortening of life of the plunger seal 12.

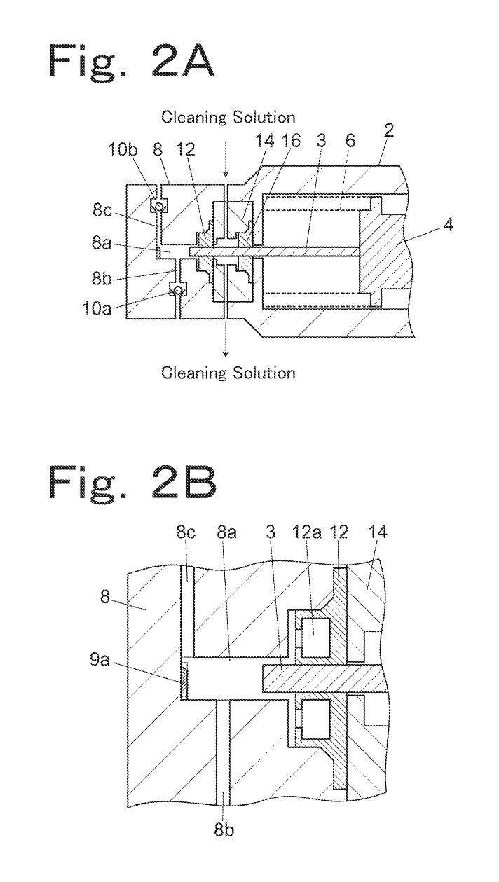

FIGS. 2A and 2B show a second example. In the example, a spacer 9a as a capacity decreasing member is provided to an innermost face of the pump chamber 8a to decrease the compression capacity. As material of the spacer 9a, PEEK resin is used. The spacer 9a has such a thickness as to fill a space between a tip end face of a plunger 3 which has reached a top dead center and the innermost face of the pump chamber 8a. Therefore, a tip end of the plunger 3 which has reached the top dead center may come in contact with the spacer 9a. In this case, however, the spacer 9a is made of the resin which can be deformed elastically, and therefore, it is possible to decrease the compression capacity without damaging the tip end of the plunger 3. Although the spacer 9a is the disk-shaped member, a notch is formed at a portion of the spacer 9a so that the spacer 9a does not close the solution discharge flow path 8c, and the notch portion is aligned with a position of the solution discharge flow path 8c.

As a method of providing the spacer 9a in the pump chamber 8a, the sleeve 9 is press-fitted in the pump chamber 8a before forming the flow paths 8b and 8c, and then the flow paths 8b and 8c are formed.

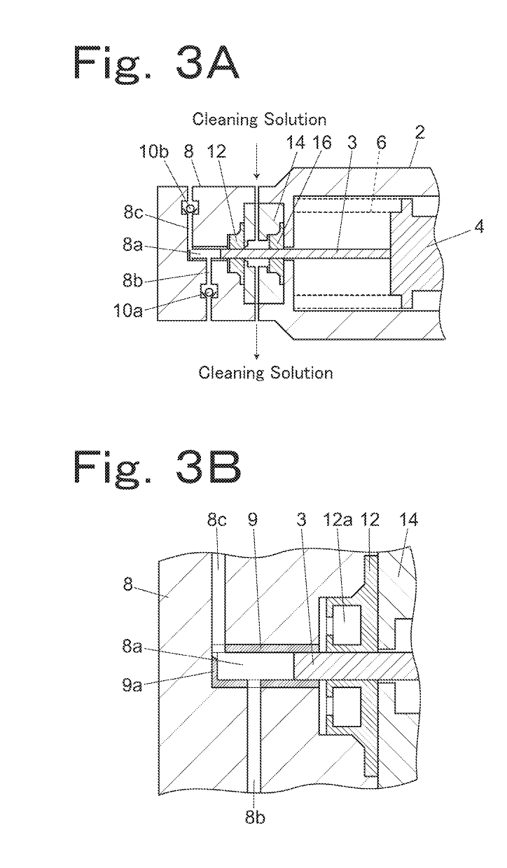

FIGS. 3A and 3B show a third example. In this example, both a sleeve 9 on an inner peripheral face of the pump chamber 8a and a spacer 9a on an innermost face of the pump chamber 8a are provided as capacity decreasing members in order to decrease the compression capacity.

As a method of providing both the sleeve 9 and the spacer 9a in the pump chamber 8a, a cylindrical member in a shape obtained by integrating the sleeve 9 and the spacer 9a is produced by using Vespel (registered trademark) which is wholly aromatic polyimide resin or PEEK resin, the cylindrical member is press-fitted so that a bottom of the cylinder is positioned on an inner side of the pump chamber 8a, and then the flow paths 8b and 8c are formed.

A plunger seal (seal member) 12 for sealing the pump chamber 8a and retaining the plunger 3 for sliding is mounted to the pump head 8. Between a syringe 2 and the pump head 8, a flange 14 is mounted. The flange 14 is in contact with the plunger seal 12 on an opposite side according to the plunger seal 12 from the pump chamber 8a to support the plunger seal 12. A hole for supporting the plunger 3 for sliding is formed at a portion of the flange 14 in contact with the plunger seal 12.

A hollow portion 12a having an opening communicating with the pump chamber 8a is provided to the plunger seal 12 so as to increase adhesion with an outer peripheral face of the plunger 3 and with an inner wall of the pump head 8 due to pressure in the pump chamber 8a.

The flange 14 has such a structure that a cleaning solution can be introduced into the flange 14 to clean the plunger 3 passing through the flange 14 and an inside of the flange 14. A cleaning seal 16 for preventing leakage of the cleaning solution introduced into the flange 14 is mounted to the flange 14 from a side of the syringe 2. The cleaning seal 16 is supported by the syringe 2.

Although the check valves 10a and 10b are provided in the pump head 8 in the solvent delivery pump shown in the example, any one of the check valves may be provided outside the pump head 8 or both of the check valves may be provided outside the pump head 8.

Although the flange 14 for allowing the cleaning of the plunger 3 or the like with the cleaning solution is mounted as the support member for supporting the plunger seal 12 in the example, the invention is not limited to this. The flange may not include the flow path for introducing and discharging the cleaning solution or may be integrated with the syringe 2.

Although the hollow portion 12a is formed in the plunger seal 12 in the example, the seal for sealing the pump chamber 8a may not be provided with the hollow portion 12a.

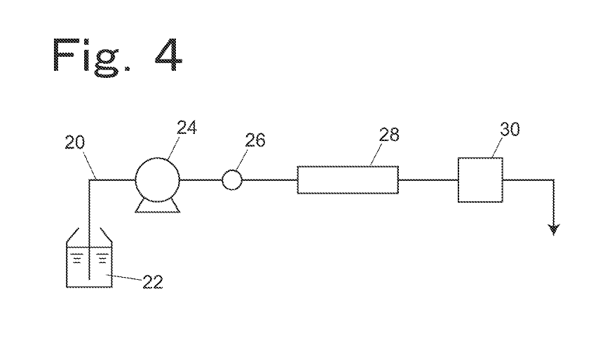

Next, an example in which the solvent delivery pump of the invention is used for a liquid chromatograph will be described by using FIG. 4.

As a solvent delivery pump 24 for circulating a mobile phase 22 in an analytical flow path 20, any one of the solution pumps of the examples described by using FIGS. 1 to 3 is used. On the analytical flow path 20, an injection port 26, an analytical column 28, and a detector 30 are disposed in this order from an upstream side. By the mobile phase 22 sent by the solvent delivery pump 24, a sample injected from the injection port 26 is introduced into the analytical column 28 and separated into ingredients and the ingredients are respectively detected by the detector 30.

Because the solvent delivery pump of the invention is used as the solvent delivery pump 24, pulsation of the solution sent by the solvent delivery pump 24 is suppressed, sending of the mobile phase 22 in the analytical flow path 20 is stabilized, and precision of analysis is increased.

The invention claimed is:

1. A solvent delivery pump comprising:
a cylindrical pump chamber an inside of which is made of metal;
a plunger inserted from a tip end into the pump chamber to reciprocate in a certain direction to increase and decrease a capacity in the pump chamber;
a capacity decreasing member made of resin and covering at least a portion of an inner wall of the pump chamber to reduce a clearance from the plunger; and
a seal member provided to a plunger insertion portion of the pump chamber to come in sliding contact with an outer peripheral face of the plunger to seal the pump chamber.

2. The solvent delivery pump according to claim 1,
wherein the capacity decreasing member is a cylindrical sleeve covering at least a portion of an inner peripheral face of the pump chamber.

3. The solvent delivery pump according to claim 1,
wherein the capacity decreasing member is a spacer disposed on an innermost face of the pump chamber to reduce a clearance from a tip end face of the plunger which has reached a top dead center.

4. The solvent delivery pump according to claim 1,
wherein the capacity decreasing member is a cylindrical sleeve covering at least a portion of an inner peripheral face of the pump chamber and a spacer disposed on an innermost face of the pump chamber to reduce a clearance from a tip end face of the plunger which has reached a top dead center.

5. The solvent delivery pump according to claim 1,
wherein material of the capacity decreasing member is wholly aromatic polyimide resin or polyether ether ketone resin.
